(19) United States
(12) Patent Application Publication
(10) Pub. No.: US 2026/0034139 A1
(43) Pub. Date: Feb. 5, 2026

(54) Use Of Piperazine Compound In Combination With Radiotherapy For Treatment Of Tumor

(71) Applicant: Kangbaida (Sichuan) Biotechnology Co., Ltd., Chendu, Sichuan (CN)

(72) Inventors: Yuqin Zhu, Chengdu, Sichuan (CN); Yonggang Wei, Chengdu, Sichuan (CN); Hongzhu Chu, Chengdu, Sichuan (CN); Fei Ye, Chengdu, Sichuan (CN); Guizhuan Su, Chengdu, Sichuan (CN); Yi Sun, Chengdu, Sichuan (CN)

(73) Assignee: Kangbaida (Sichuan) Biotechnology Co., Ltd., Chendu, Sichuan (CN)

(21) Appl. No.: 19/099,966

(22) PCT Filed: Jul. 31, 2023
(86) PCT No.: PCT/CN2023/110230

§ 371 Date: Jan. 30, 2025

(30) Foreign Application Priority Data
Aug. 9, 2022 (CN) 202210949131.9

(51) Int. Cl. A61K 31/5377; A61K 31/501; A61K 31/506; A61K 31/519; A61N 5/00; A61P 35/00

(52) U.S. Cl. CPC A61K 31/5377; A61K 31/501; A61K 31/506; A61K 31/519; A61N 5/00; A61P 35/00

(57) ABSTRACT

The present application relates to the use of a piperazine compound in combination with radiotherapy for the treatment of tumors.

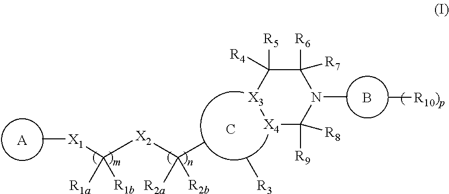

### TECHNICAL FIELD

The present application relates to the use of a piperazine compound in combination with radiotherapy for the treatment of tumors.

### BACKGROUND ART

Radiotherapy is one of the important methods for the comprehensive treatment of malignant tumors, and the neoadjuvant therapy based on conventional long-term radiotherapy has become a standard regimen for treating various malignant tumors. However, long-term clinical studies have found that the responses of patients with malignant tumors to radiotherapy vary greatly. Although some patients can achieve complete pathological remission at the end of a treatment cycle, there are still some patients who cannot benefit from long-term treatment, and a few even develop worsening progress. Therefore, it is clinically significant to improve the therapeutic effect of tumors by combining small molecule anti-tumor compounds and radiotherapy.

### SUMMARY

The PCT Application PCT/CN 2022/094124 by the present applicant describes the use of a piperazine derivative in the preparation of a medicament for the treatment of cancer, wherein the compounds described in the specification have a high selectivity and significant inhibitory activity against PARP7. In subsequent studies, it was unexpectedly found that the piperazine derivative can significantly improve the anti-tumor effect of radiotherapy.

Accordingly, the present invention aims to provide the use of a piperazine compound in combination with radiotherapy for the treatment of tumors.

One or more embodiments of the present invention provide the use of a compound of general formula (I), or a pharmaceutically acceptable salt, a stereoisomer or a deuterated compound thereof as an active ingredient in combination with radiotherapy for the treatment of tumors:

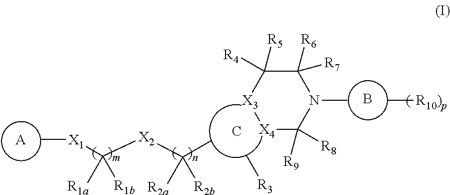

wherein
X₁ is NH, O or 4- to 6-membered heterocycle containing 1 to 3 heteroatoms selected from N and O;
X₂ is O or a single bond;
X₃ and X₄ are each independently C or N;
R₁ₐ, and R_{1b} are each independently H, D or C₁₋₆ alkyl; or R₁ₐ, and R_{1b} together with the carbon atom connected thereto form 3- to 5-membered cycloalkyl;
R₂ₐ and R_{2b} are each independently H, D or C₁₋₆ alkyl; or R₂ₐ and R_{2b} together with the carbon atom connected thereto form 3- to 5-membered cycloalkyl;
R₃ is H, D, C₁₋₆ alkyl, halogen or cyano, wherein the C₁₋₆ alkyl is optionally substituted with 1 to 3 halogens;
R₄ and R₅ are each independently H, D or C₁₋₆ alkyl; or R₄ and R₅ together with the carbon atom connected thereto form 3- to 5-membered cycloalkyl;
R₆ and R₇ are each independently H, D or C₁₋₆ alkyl; or R₆ and R₇ together with the carbon atom connected thereto form C═O;
R₈ and R₉ are each independently H, D or C₁₋₆ alkyl; or R₈ and R₉ together with the carbon atom connected thereto form C═O; or R₈ and R₉ together with the carbon atom connected thereto form 3- to 5-membered cycloalkyl;
each R₁₀ is independently C₁₋₆alkyl, C₁₋₆ alkoxy, CONR₁₀ₐR_{10b}, halogen, cyano, S(O)₂R_{10c}, SR_{10d} or 3- to 5-membered cycloalkyl, wherein the C₁₋₆ alkyl and C₁₋₆ alkoxy are optionally substituted with 1 to 3 halogens;
R₁₀ₐ, R_{10b}, R_{10c} and R_{10d} are each independently H, D or C₁₋₆ alkyl;
A is

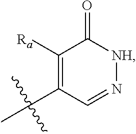

and Rₐ is C₁₋₆ alkyl, C₃₋₅ cycloalkyl, halogen or cyano, wherein the C₁₋₆alkyl is optionally substituted with 1 to 3 halogens;
B is 5- to 10-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3;
n is 0, 1, 2 or 3; and
p is 0, 1, 2 or 3.

One or more embodiments of the present invention provide the use of a compound of general formula (I), or a pharmaceutically acceptable salt, a stereoisomer or a deuterated compound thereof as an active ingredient in combination with radiotherapy for the treatment of tumors, wherein
X₁ is NH, O or 4- to 6-membered heterocycle containing 1 to 3 heteroatoms selected from N and O;
X₂ is O or a single bond;
X₃ and X₄ are each independently C or N;
R₁ₐ, and R_{1b} are each independently H, D or C₁₋₆ alkyl;
R₂ₐ and R_{2b} are each independently H, D or C₁₋₆ alkyl; or R₂ₐ and R_{2b} together with the carbon atom connected thereto form 3- to 5-membered cycloalkyl;
R₃ is H, D, C₁₋₆ alkyl, halogen or cyano, wherein the C₁₋₆alkyl is optionally substituted with 1 to 3 halogens;
R₄ and R₅ are each independently H, D or C₁₋₆ alkyl; or R₄ and R₅ together with the carbon atom connected thereto form 3- to 5-membered cycloalkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
each R₁₀ is independently C₁₋₆alkyl, C₁₋₆ alkoxy, CONR₁₀ₐR_{10b}, halogen, cyano, S(O)₂R_{10c}, SR_{10d} or 3- to 5-membered cycloalkyl, wherein the C₁₋₆ alkyl and C₁₋₆ alkoxy are optionally substituted with 1 to 3 halogens;
R₁₀ₐ, R_{10b}, R_{10c} and R_{10d} are each independently H, D or C₁₋₆ alkyl;
A is

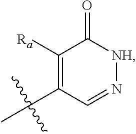

and Rₐ is C₁₋₆ alkyl, C₃₋₅ cycloalkyl, halogen or cyano, wherein the C₁₋₆ alkyl is optionally substituted with 1 to 3 halogens;
B is 5- to 6-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3;
n is 0, 1, 2 or 3; and
p is 0, 1, 2 or 3.

One or more embodiments of the present invention provide the use of a compound of general formula (I-1), or a pharmaceutically acceptable salt, a stereoisomer or a deuterated compound thereof as an active ingredient in combination with radiotherapy for the treatment of tumors:

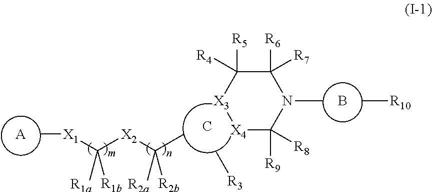

wherein
X₁ is NH or 4- to 6-membered heterocycle containing 1 to 3 heteroatoms selected from N and 0;
X₂ is O;
X₃ and X₄ are each independently C or N;
R₁ₐ, and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is

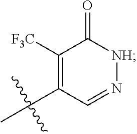 [SMILES: *c1cn[nH]c(=O)c1C(F)(F)F]

B is 5- to 6-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3; and
n is 0, 1, 2 or 3.

One or more embodiments of the present invention provide the use of a compound of general formula (I-2), or a pharmaceutically acceptable salt, a stereoisomer or a deuterated compound thereof as an active ingredient in combination with radiotherapy for the treatment of tumors:

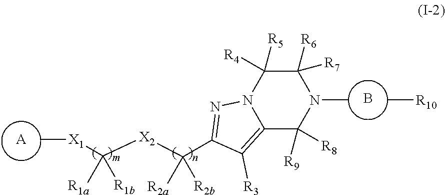

wherein
X₁ is NH;
X₂ is O;
R₁ₐ, and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is

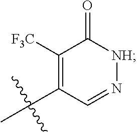 [SMILES: *c1cn[nH]c(=O)c1C(F)(F)F]

B is 5- to 6-membered carbocycle or heterocycle, wherein the heterocycle contains 1 to 3 heteroatoms selected from N, O and S;
m is 1, 2 or 3; and
n is 0, 1, 2 or 3.

One or more embodiments of the present invention provide the use of a compound of general formula (I-2), or a pharmaceutically acceptable salt, a stereoisomer or a deuterated compound thereof as an active ingredient in combination with radiotherapy for the treatment of tumors, wherein
X₁ is NH;
X₂ is O;
R₁ₐ, and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, cyano or SR_{10d}, wherein the C₁₋₆alkyl is optionally substituted with 1 to 3 halogens;
R_{10d} is H, D or C₁₋₆ alkyl;
A is

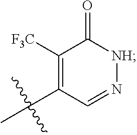 [SMILES: *c1cn[nH]c(=O)c1C(F)(F)F]

B is 5- to 6-membered carbocycle or heterocycle, wherein the heterocycle contains 1 to 3 heteroatoms selected from N, O and S;
m is 1, 2 or 3; and
n is 0, 1, 2 or 3.

One or more embodiments of the present invention provide the use of a compound of general formula (I-2), or a pharmaceutically acceptable salt, a stereoisomer or a deuterated compound thereof as an active ingredient in combination with radiotherapy for the treatment of tumors, wherein
X₁ is selected from NH;
X₂ is selected from 0;
R₁ₐ, and R_{1b} are each independently selected from H, D or C₁₋₆ alkyl;
R₂ₐ and R_{2b} are each independently selected from H, D or C₁₋₆ alkyl;
R₃ is H, D, C₁₋₆ alkyl or halogen, wherein the C₁₋₆alkyl is optionally substituted with 1 to 3 halogens;
R₄ and R₅ are each independently H or D;
R₆ and R₇ are each independently H or D;
R₈ and R₉ are each independently H or D;
R₁₀ is CF₃ or SR_{10d};
R_{10d} is H, D or C₁₋₆ alkyl;
A is

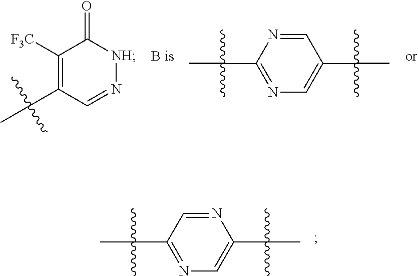 [SMILES: *c1cn[nH]c(=O)c1C(F)(F)F ; *c1cnc(*)nc1 ; *c1cnc(*)cn1]

m is 1, 2 or 3; and
n is 0, 1, 2 or 3.

One or more embodiments of the present invention provide the use of a compound of general formula (I-2), or a pharmaceutically acceptable salt, a stereoisomer or a deuterated compound thereof as an active ingredient in combination with radiotherapy for the treatment of tumors, wherein
X₁ is selected from NH;
X₂ is selected from 0;
R₁ₐ and R_{1b} are each independently selected from H, D or C₁₋₃ alkyl;
R₂ₐ and R_{2b} are each independently selected from H, D or C₁₋₃ alkyl;
R₃ is selected from H, D or CF₃;
R₄ and R₅ are each independently selected from H or D;
R₆ and R₇ are each independently selected from H or D;
R₈ and R₉ are each independently selected from H or D;
R₁₀ is CF₃;
A is

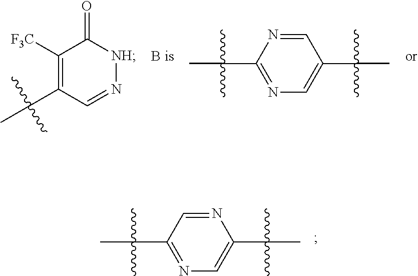 [SMILES: *c1cn[nH]c(=O)c1C(F)(F)F ; *c1cnc(*)nc1 ; *c1cnc(*)cn1]

m is 1, 2 or 3; and
n is 0, 1 or 2.

One or more embodiments of the present invention provide the use of a compound of general formula (I-3), or a pharmaceutically acceptable salt, a stereoisomer or a deuterated compound thereof as an active ingredient in combination with radiotherapy for the treatment of tumors:

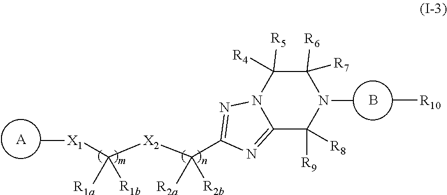

wherein
X₁ is NH or 4- to 6-membered heterocycle containing 1 to 3 heteroatoms selected from N and 0;
X₂ is O;
R₁ₐ, and R_{1b} are each independently H, D or C₁₋₆ alkyl;
R₂ₐ and R_{2b} are each independently H, D or C₁₋₆ alkyl;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is

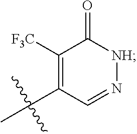 [SMILES: *c1cn[nH]c(=O)c1C(F)(F)F]

B is 5- to 6-membered carbocycle or heterocycle, wherein the heterocycle contains 1 to 3 heteroatoms selected from N, O and S;
m is 1, 2 or 3; and
n is 0, 1, 2 or 3.

One or more embodiments of the present invention provide the use of a compound of general formula (I-3), or a pharmaceutically acceptable salt, a stereoisomer or a deuterated compound thereof as an active ingredient in combination with radiotherapy for the treatment of tumors, wherein
X₁ is NH or 4- to 6-membered heterocycle containing 1 to 3 heteroatoms selected from N and 0;
X₂ is O;
R₁ₐ, and R_{1b} are each independently H, D or C₁₋₃ alkyl;
R₂ₐ and R_{2b} are each independently H, D or C₁₋₃ alkyl;
R₄ and R₅ are each independently H, D or C₁₋₃ alkyl;
R₆ and R₇ are each independently H, D or C₁₋₃ alkyl;
R₈ and R₉ are each independently H, D or C₁₋₃ alkyl; or R₈ and R₉ together with the carbon atom connected thereto form C═O;
R₁₀ is C₁₋₆ alkyl, cyano or SR_{10d}, wherein the C₁₋₆alkyl is optionally substituted with 1 to 3 halogens;
R_{10d} is H, D or C₁₋₆ alkyl;
A is

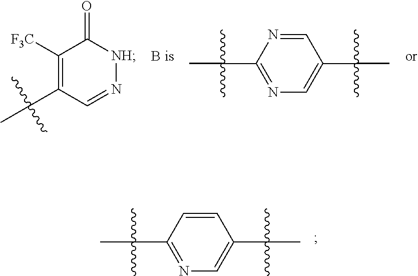 [SMILES: *c1cn[nH]c(=O)c1C(F)(F)F ; *c1cnc(*)nc1 ; *c1ccc(*)nc1]

m is 1, 2 or 3; and
n is 0, 1 or 2.

One or more embodiments of the present invention provide the use of a compound of general formula (I-3), or a pharmaceutically acceptable salt, a stereoisomer or a deuterated compound thereof as an active ingredient in combination with radiotherapy for the treatment of tumors, wherein
X₁ is NH;
X₂ is O;
R₁ₐ, and R_{1b} are each independently H, D or C₁₋₃ alkyl;
R₂ₐ and R_{2b} are each independently H or D;
R₄ and R₅ are each independently H or D;
R₆ and R₇ are each independently H or D;
R₈ and R₉ are each independently H or D;
R₁₀ is CF₃;
A is

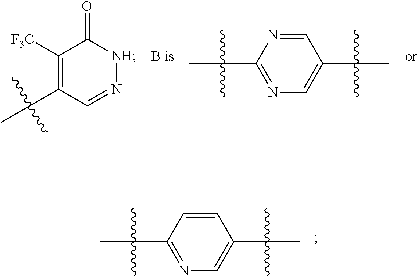 [SMILES: *c1cn[nH]c(=O)c1C(F)(F)F ; *c1cnc(*)nc1 ; *c1ccc(*)nc1]

m is 1, 2 or 3; and
n is 0, 1 or 2.

In one or more embodiments of the present invention, the active ingredient is selected from compounds as shown below, or a pharmaceutically acceptable salt, a stereoisomer, or a deuterated compound thereof:

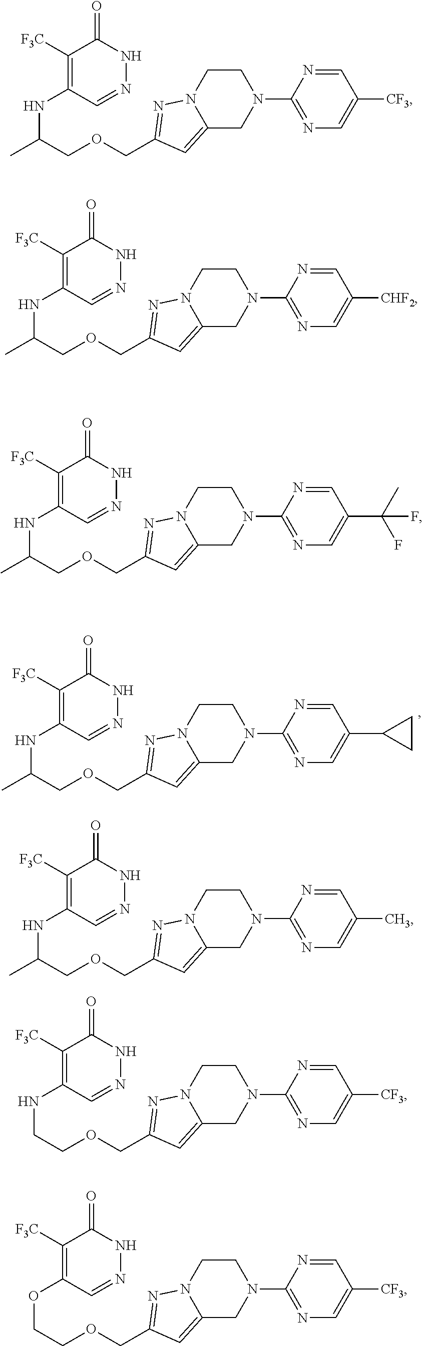

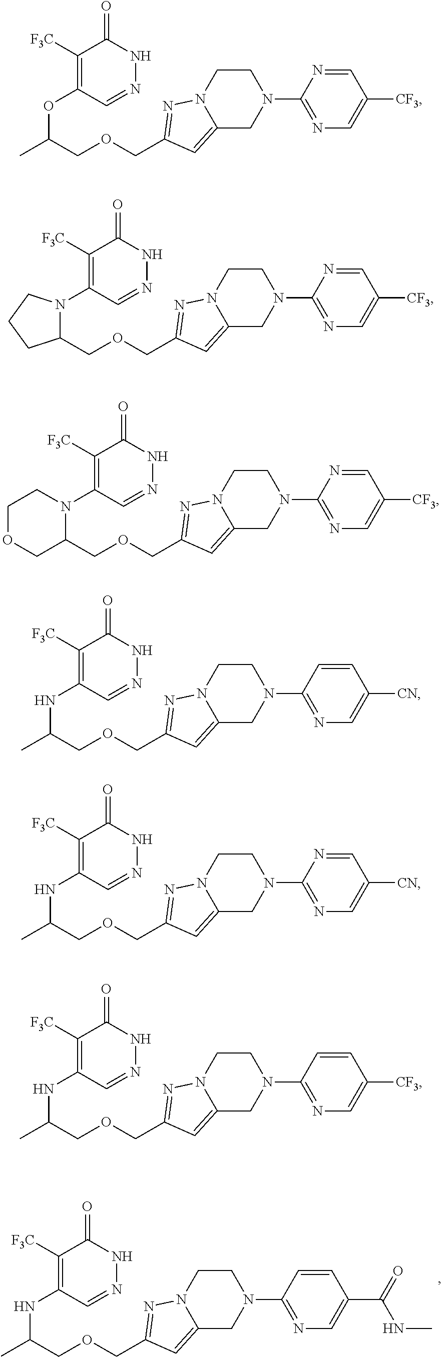

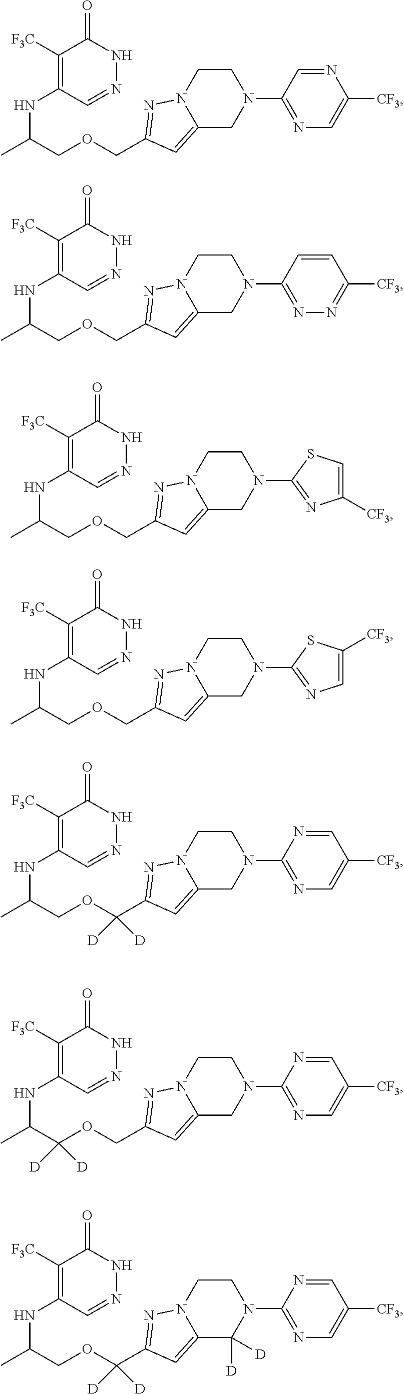

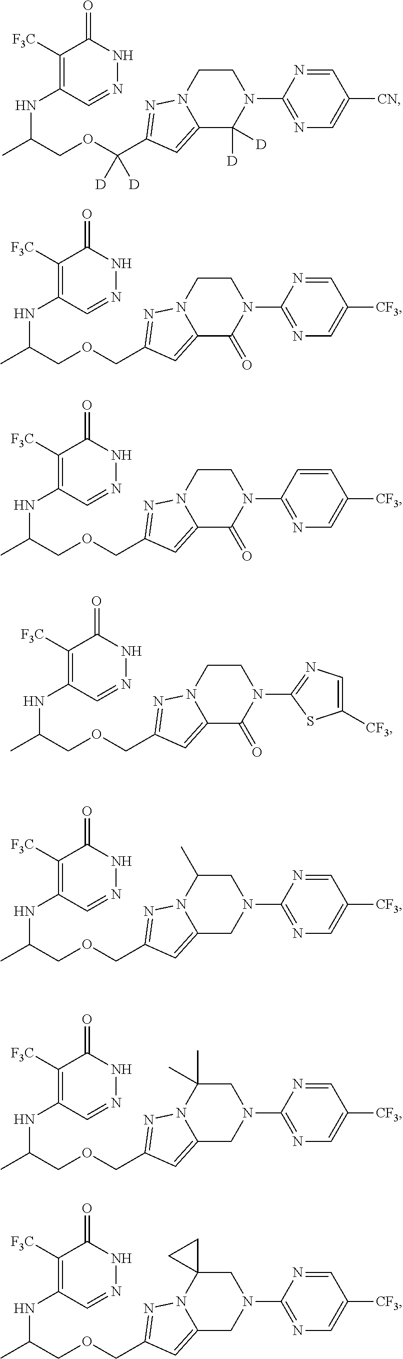

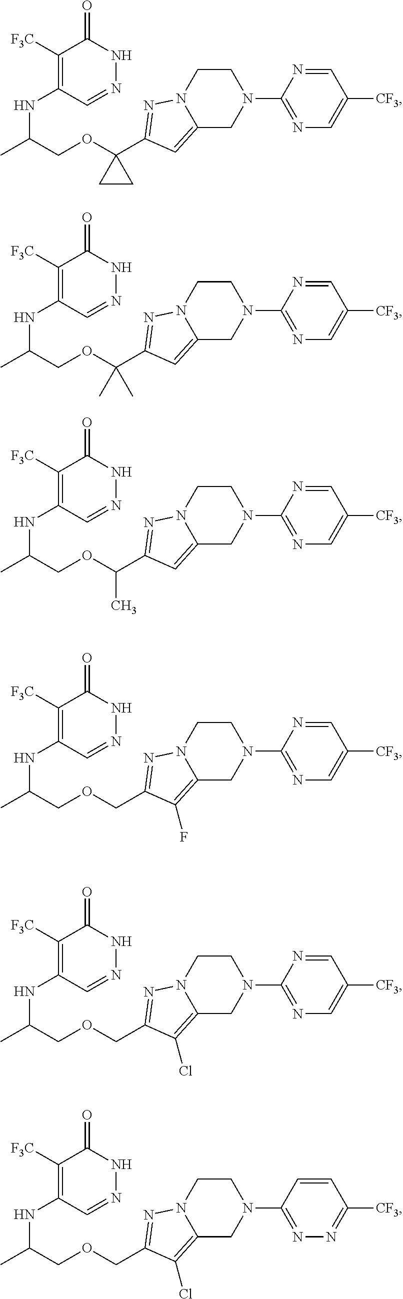

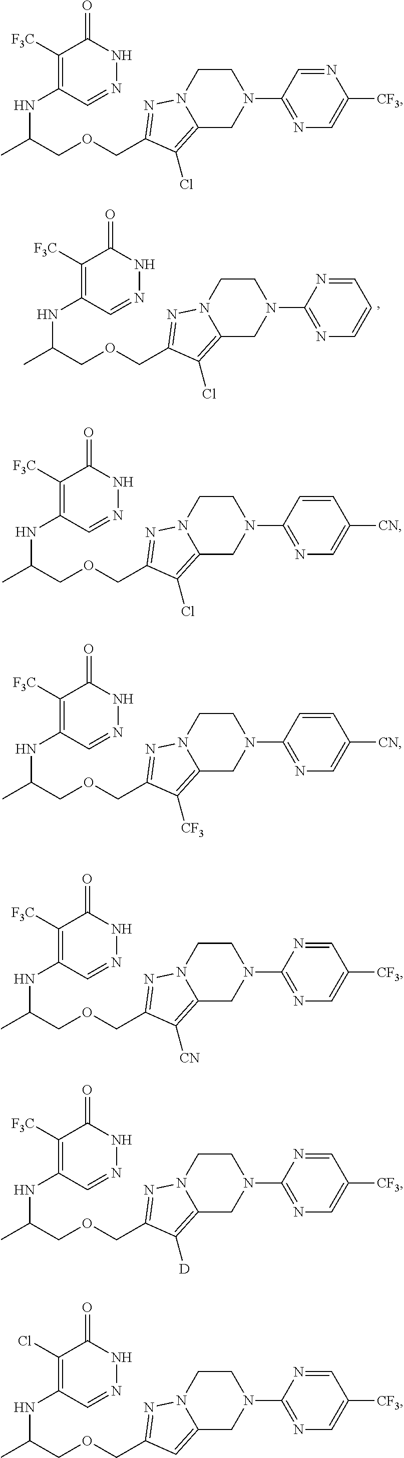

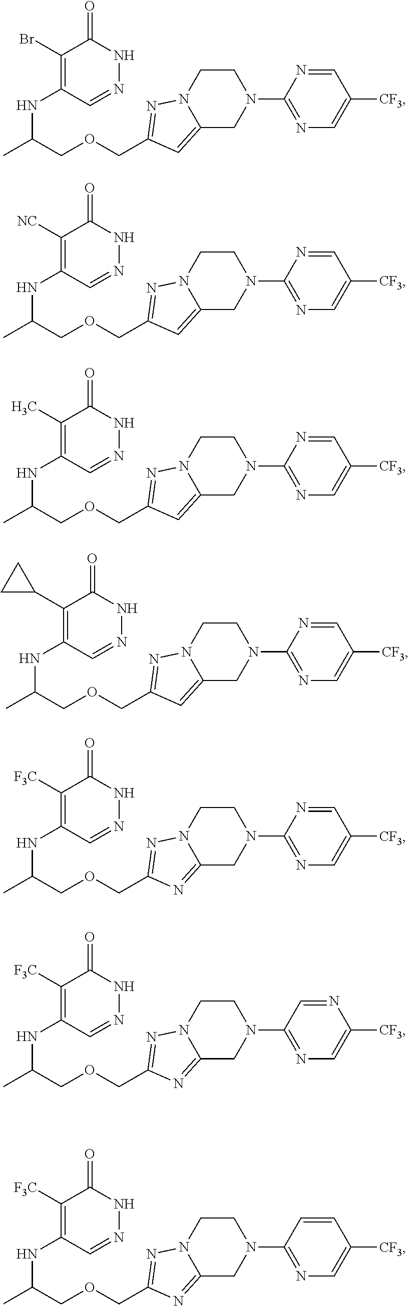

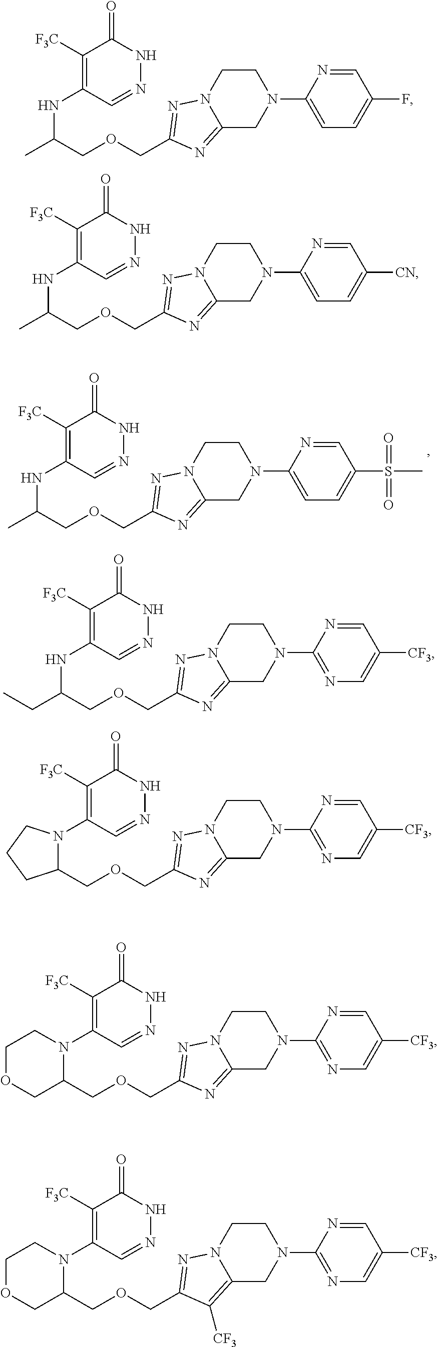

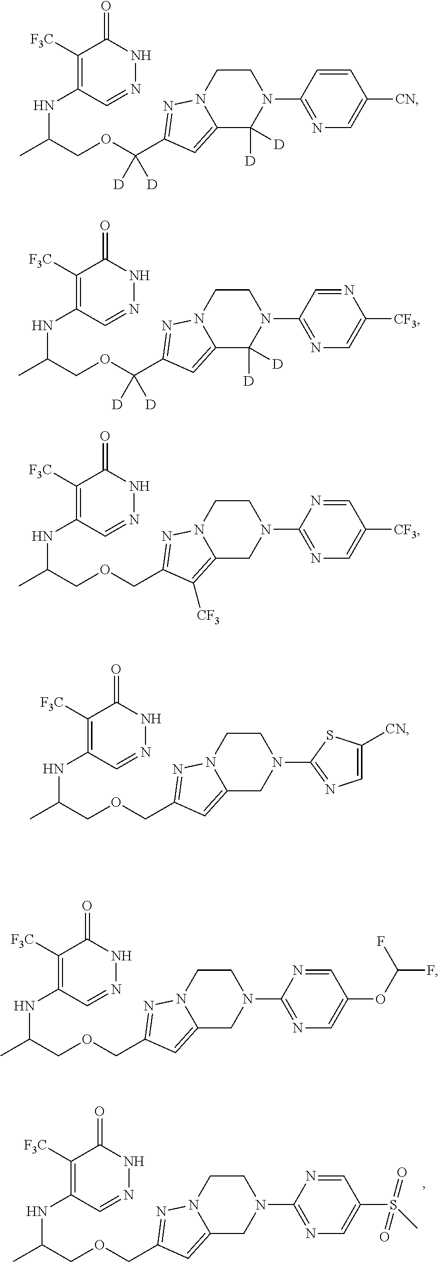

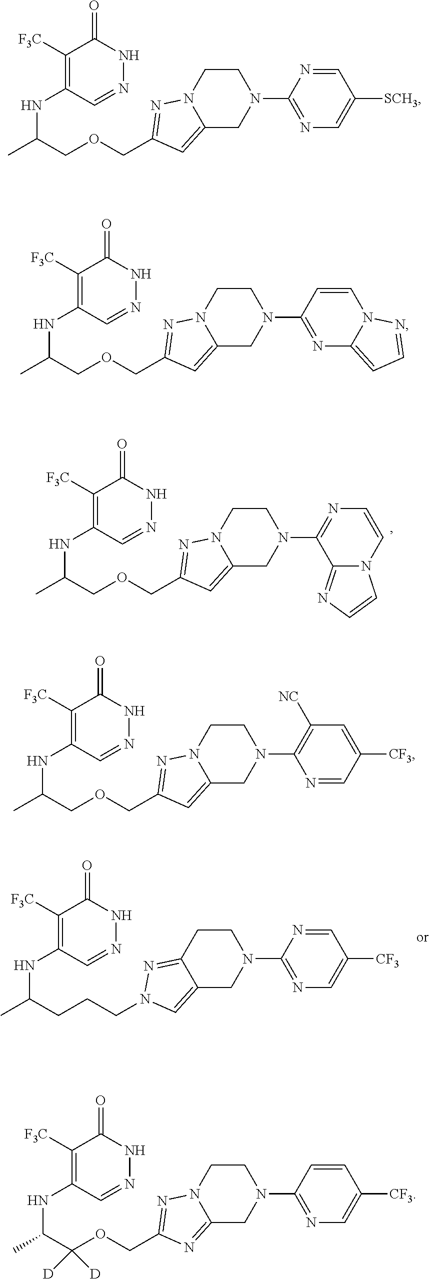 [SMILES: CSc1cnc(N2CCn3nc(COCC(C)Nc4cn[nH]c(=O)c4C(F)(F)F)cc3C2)nc1 ; CC(COCc1cc2n(n1)CCN(c1ccn3nccc3n1)C2)Nc1cn[nH]c(=O)c1C(F)(F)F ; CC(COCc1cc2n(n1)CCN(c1nccn3ccnc13)C2)Nc1cn[nH]c(=O)c1C(F)(F)F ; CC(COCc1cc2n(n1)CCN(c1ncc(C(F)(F)F)cc1C#N)C2)Nc1cn[nH]c(=O)c1C(F)(F)F ; CC(CCCn1cc2c(n1)CCN(c1ncc(C(F)(F)F)cn1)C2)Nc1cn[nH]c(=O)c1C(F)(F)F ; [2H]C([2H])(OCc1nc2n(n1)CCN(c1ccc(C(F)(F)F)cn1)C2)[C@H](C)Nc1cn[nH]c(=O)c1C(F)(F)F]

In one or more embodiments of the present application, when the active ingredient is applied in combination with radiotherapy, there is a synergistic effect.

In one or more embodiments of the present application, the active ingredient is contained in a pharmaceutical composition further comprising one or more pharmaceutically acceptable excipients, diluents or carriers.

In one or more embodiments of the present application, the total daily dosage of the active ingredient is selected from 50-1500 mg, preferably 100-1000 mg, and more preferably 400-800 mg, as measured by the amount of free base.

In one or more embodiments of the present application, the active ingredient and the radiotherapy are administered simultaneously.

In one or more embodiments of the present application, the active ingredient and the radiotherapy are administered sequentially, e.g., the active ingredient is administered first, followed by the radiotherapy, alternatively, the radiotherapy is administered first, followed by the active ingredient. The administration intervals may be in hours, days, weeks, or months.

In one or more embodiments of the present application, the active ingredient is given orally at a frequency of thrice daily, twice daily, once daily, once every two days, once every three days, once every four days, once every five days, once every six days, once a week, once every two weeks, once every three weeks, or once every four weeks, preferably twice daily or once daily.

In one or more embodiments of the present application, the tumor is selected from solid tumor.

In one or more embodiments of the present application, the solid tumor is selected from non-small cell lung cancer, head and neck squamous carcinoma, esophageal squamous carcinoma, hormone-receptor-positive (HR+) breast cancer, advanced solid tumor with PARP7 expansion, colon cancer or lung cancer, preferably colon cancer or lung cancer, and more preferably colon cancer.

### Advantages of the Present Invention

The compound \textcircled{1} in combination with radiotherapy has an anti-tumor effect significantly superior to that of radiotherapy alone, and is also well-tolerated and safe. It is shown that when the active ingredient of the present invention is applied in combination with radiotherapy, there is a significant synergistic effect.

### BRIEF DESCRIPTION OF THE DRAWINGS

FIG. 1 shows the structural formula of the compound \textcircled{1}.

FIG. 2 shows growth curves of tumor volume of tumor-bearing mice.

FIG. 3 shows percent body weight change of tumor-bearing mice.

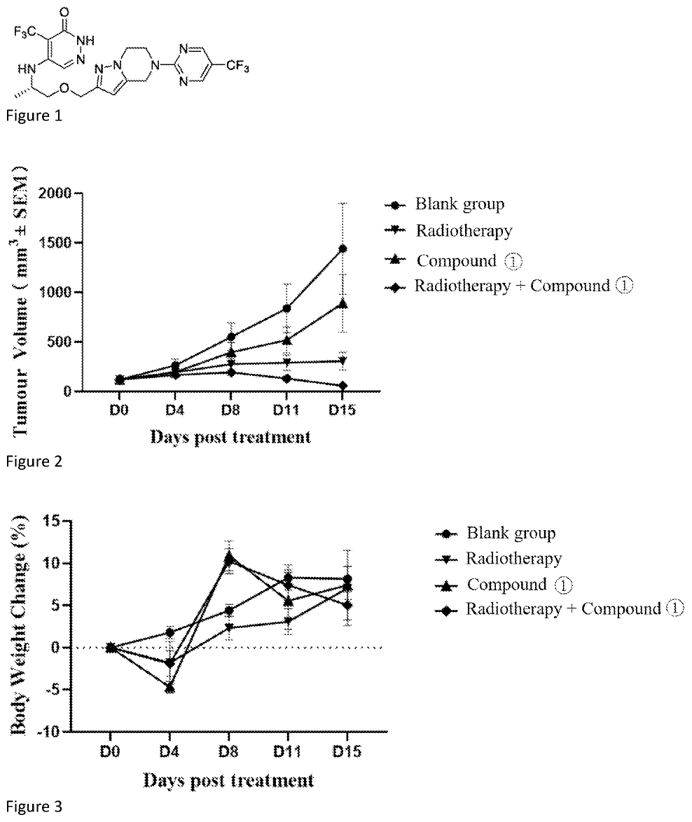

### DETAILED DESCRIPTION OF EMBODIMENTS

The implementation process and beneficial effects of the present invention are described in detail below by way of specific examples, which are intended to help readers better understand the essence and characteristics of the present invention but not to limit the scope of implementation of the present invention.

The present invention will be described in further detail below with reference to the accompanying drawings.

The compound \textcircled{1} in the examples is compound 1 of PCT application PCT/CN2022/094124 and is prepared according to the preparation method therefor.

### Efficacy Test in CT26 Mouse Tumor-Bearing Model

### 1. Experimental Steps

### 1.1 Cell Culture

Colon cancer cells CT26 were purchased from ATCC for culture under conditions of a DMEM medium supplemented with 10% fetal bovine serum and 1% penicillin and streptomycin, in a cell incubator containing 5% CO₂ at 37° C. The cells in an exponential growth phase were digested with trypsin, and the cells were collected and counted, followed by inoculation.

### 1.2 Subcutaneously-Transplanted Tumor Construction

Female BALB/c mice were selected to constitute a CT26 subcutaneously-transplanted tumor model by subcutaneous injection of tumor cells. When the average tumor volume reached 120-130 mm³, 32 animals were screened for enrollment. The animals were randomly divided into 4 groups based on the tumor volume, namely the blank group, the compound \textcircled{1} group, the irradiated group, and the combined compound \textcircled{1} and irradiation group, with 8 animals per group. The day of the grouping was set as day 0 of the trial (PG-DO). Each irradiated group was given an irradiation dose of 2 Gy for 5 consecutive days starting from D1, and compound ((125 mpk, BID) administration began on day 1 (D1).

### 2. Assay Method

### 2.1 Tumor Volume

The diameter of tumors was measured twice a week using a vernier caliper to calculate the volume of tumors, and a tumor growth curve was plotted. The calculation formula of the tumor volume (V) is:

V=½×a×b²,

where a and b represent the long and short diameters of the tumor, respectively.

### 2.2 Body Weight of Mice

Mice were weighed at least twice a week during a drug treatment cycle.

### 3. Experimental Results

As shown in FIG. 2 and FIG. 3, the anti-tumor effect of compound \textcircled{1} in combination with radiotherapy is significantly superior to that of radiotherapy alone, and is also well-tolerated and safe. It is shown that when the active ingredient of the present invention is applied in combination with radiotherapy, there is a significant synergistic effect.

Specific embodiments have been described in detail in the specification of the present invention. A person skilled in the art should recognize that the embodiments described above are exemplary and cannot be construed as limiting the present invention. Additionally, a person skilled in the art can make several improvements and modifications to the present invention without departing from the principle of the present invention, and the technical solutions obtained based on these improvements and modifications also fall within the scope of protection of the claims of the present invention.

1. A method for treating tumors, comprising administering a therapeutically effective amount of a compound of formula (I), or a pharmaceutically acceptable salt, a stereoisomer or a deuterated compound thereof as an active ingredient in combination with radiotherapy: 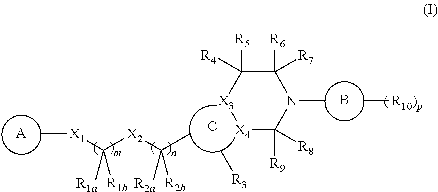
wherein
X₁ is NH, O or 4- to 6-membered heterocycle containing 1 to 3 heteroatoms selected from N and O;
X₂ is O or a single bond;
X₃ and X₄ are each independently C or N;
R₁ₐ and R_{1b} are each independently H, D or C₁₋₆ alkyl; or R₁ₐ and R_{1b} together with the carbon atom connected thereto form 3- to 5-membered cycloalkyl;
R₂ₐ and R_{2b} are each independently H, D or C₁₋₆ alkyl; or R₂ₐ and R_{2b} together with the carbon atom connected thereto form 3- to 5-membered cycloalkyl;
R₃ is H, D, C₁₋₆ alkyl, halogen or cyano, wherein the C₁₋₆ alkyl is optionally substituted with 1 to 3 halogens;
R₄ and R₅ are each independently H, D or C₁₋₆ alkyl; or R₄ and R₅ together with the carbon atom connected thereto form 3- to 5-membered cycloalkyl;
R₆ and R₇ are each independently H, D or C₁₋₆ alkyl; or R₆ and R₇ together with the carbon atom connected thereto form C═O;
R₈ and R₉ are each independently H, D or C₁₋₆ alkyl; or R₈ and R₉ together with the carbon atom connected thereto form C═O; or R₈ and R₉ together with the carbon atom connected thereto form 3- to 5-membered cycloalkyl;
each R₁₀ is independently C₁₋₆ alkyl, C₁₋₆ alkoxy, CONR₁₀ₐR_{10b}, halogen, cyano, S(O)₂R_{10c}, SR_{10d} or 3- to 5-membered cycloalkyl, wherein the C₁₋₆ alkyl and C₁₋₆ alkoxy are optionally substituted with 1 to 3 halogens;
R₁₀ₐ, R_{10b}, R_{10c} and R_{10d} are each independently H, D or C₁₋₆ alkyl;
A is 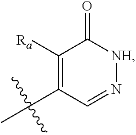
and Rₐ is C₁₋₆ alkyl, C₃₋₅ cycloalkyl, halogen or cyano, wherein the C₁₋₆ alkyl is optionally substituted with 1 to 3 halogens;
B is 5- to 10-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3;
n is 0, 1, 2 or 3; and
p is 0, 1, 2 or 3.

2. The method according to claim 1, wherein the active ingredient is a compound of formula (I), or a pharmaceutically acceptable salt, a stereoisomer, or a deuterated compound thereof, wherein
X₁ is NH, O or 4- to 6-membered heterocycle containing 1 to 3 heteroatoms selected from N and O;
X₂ is O or a single bond;
X₃ and X₄ are each independently C or N;
R₁ₐ and R_{1b} are each independently H, D or C₁₋₆ alkyl;
R₂ₐ and R_{2b} are each independently H, D or C₁₋₆ alkyl; or R₂ₐ and R_{2b} together with the carbon atom connected thereto form 3- to 5-membered cycloalkyl;
R₃ is H, D, C₁₋₆ alkyl, halogen or cyano, wherein the C₁₋₆ alkyl is optionally substituted with 1 to 3 halogens;
R₄ and R₅ are each independently H, D or C₁₋₆ alkyl; or R₄ and R₅ together with the carbon atom connected thereto form 3- to 5-membered cycloalkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
each R₁₀ is independently C₁₋₆ alkyl, C₁₋₆ alkoxy, CONR₁₀ₐR_{10b}, halogen, cyano, S(O)₂R_{10c}, SR_{10d} or 3- to 5-membered cycloalkyl, wherein the C₁₋₆ alkyl and C₁₋₆ alkoxy are optionally substituted with 1 to 3 halogens;
R₁₀ₐ, R_{10b}, R_{10c} and R_{10d} are each independently H, D or C₁₋₆ alkyl;
A is 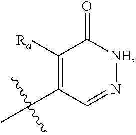
and Rₐ is C₁₋₆ alkyl, C₃₋₅ cycloalkyl, halogen or cyano, wherein the C₁₋₆ alkyl is optionally substituted with 1 to 3 halogens;
B is 5- to 6-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3;
n is 0, 1, 2 or 3; and
p is 0, 1, 2 or 3.

3. The use-method according to claim 2, wherein the active ingredient is a compound of formula (I-1), or a pharmaceutically acceptable salt, a stereoisomer, or a deuterated compound thereof, wherein 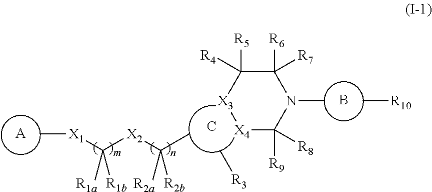
wherein
X₁ is NH or 4- to 6-membered heterocycle containing 1 to 3 heteroatoms selected from N and O;
X₂ is O;
X₃ and X₄ are each independently C or N;
R₁ₐ and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆ alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is 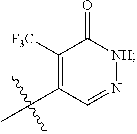 [SMILES: *c1cn[nH]c(=O)c1C(F)(F)F]
B is 5- to 6-membered carbocycle or heterocycle, wherein the heterocycle contains 1 to 3 heteroatoms selected from N, O and S;
C is 5- to 6-membered heterocycle containing 1 to 3 heteroatoms of N;
m is 1, 2 or 3; and
n is 0, 1, 2 or 3.

4. The use-method according to claim 3, wherein the active ingredient is a compound of formula (I-2), or a pharmaceutically acceptable salt, a stereoisomer, or a deuterated compound thereof, wherein 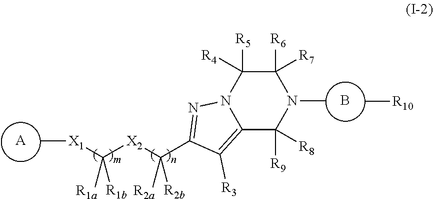
wherein
X₁ is NH;
X₂ is O;
R₁ₐ and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆ alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is 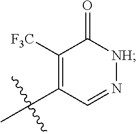 [SMILES: *c1cn[nH]c(=O)c1C(F)(F)F]
B is 5- to 6-membered carbocycle or heterocycle, wherein the heterocycle contains 1 to 3 heteroatoms selected from N, O and S;
m is 1, 2 or 3; and
n is 0, 1, 2 or 3.

5. The use-method according to claim 4, wherein the active ingredient is a compound of formula (I-2), or a pharmaceutically acceptable salt, a stereoisomer, or a deuterated compound thereof, wherein
X₁ is NH;
X₂ is O;
R₁ₐ and R_{1b} are each independently H, D or C₁₋₆ alkyl;
R₂ₐ and R_{2b} are each independently H, D or C₁₋₆ alkyl;
R₃ is H, D, C₁₋₆ alkyl or halogen, wherein the C₁₋₆ alkyl is optionally substituted with 1 to 3 halogens;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, cyano or SR_{10d}, wherein the C₁₋₆ alkyl is optionally substituted with 1 to 3 halogens;
R_{10d} is H, D or C₁₋₆ alkyl;
A is 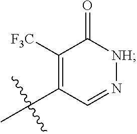 [SMILES: *c1cn[nH]c(=O)c1C(F)(F)F]
B is 5- to 6-membered carbocycle or heterocycle, wherein the heterocycle contains 1 to 3 heteroatoms selected from N, O and S;
m is 1, 2 or 3; and
n is 0, 1, 2 or 3.

6. The use-method according to claim 5, wherein the active ingredient is a compound of formula (I-2), or a pharmaceutically acceptable salt, a stereoisomer, or a deuterated compound thereof, wherein
X₁ is selected from NH;
X₂ is selected from O;
R₁ₐ and R_{1b} are each independently selected from H, D or C₁₋₆ alkyl;
R₂ₐ and R_{2b} are each independently selected from H, D or C₁₋₆ alkyl;
R₃ is H, D, C₁₋₆ alkyl or halogen, wherein the C₁₋₆ alkyl is optionally substituted with 1 to 3 halogens;
R₄ and R₅ are each independently H or D;
R₆ and R₇ are each independently H or D;
R₈ and R₉ are each independently H or D;
R₁₀ is CF₃ or SR_{10d};
R_{10d} is H, D or C₁₋₆ alkyl;
A is 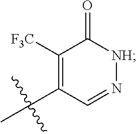 [SMILES: *c1cn[nH]c(=O)c1C(F)(F)F]
B is 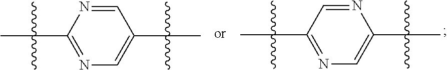 [SMILES: *c1cnc(*)nc1 ; *c1cnc(*)cn1]
m is 1, 2 or 3; and
n is 0, 1, 2 or 3.

7. The method according to claim 6, wherein the active ingredient is a compound of formula (I-2), or a pharmaceutically acceptable salt, a stereoisomer, or a deuterated compound thereof, wherein
X₁ is selected from NH;
X₂ is selected from O;
R₁ₐ and R_{1b} are each independently selected from H, D or C₁₋₃ alkyl;
R₂ₐ and R_{2b} are each independently selected from H, D or C₁₋₃ alkyl;
R₃ is selected from H, D or CF₃;
R₄ and R₅ are each independently selected from H or D;
R₆ and R₇ are each independently selected from H or D;
R₈ and R₉ are each independently selected from H or D;
R₁₀ is CF₃;
A is 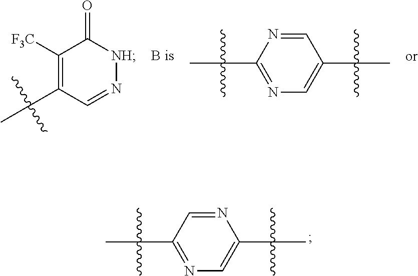 [SMILES: *c1cn[nH]c(=O)c1C(F)(F)F ; *c1cnc(*)nc1 ; *c1cnc(*)cn1]
m is 1, 2 or 3; and
n is 0, 1 or 2.

8. The method according to claim 1, wherein the active ingredient is a compound of formula (I-3), or a pharmaceutically acceptable salt, a stereoisomer, or a deuterated compound thereof, wherein 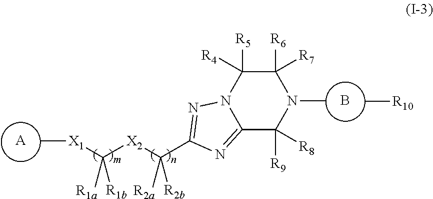
wherein
X₁ is NH or 4- to 6-membered heterocycle containing 1 to 3 heteroatoms selected from N and O;
X₂ is O;
R₁ₐ and R_{1b} are each independently H, D or C₁₋₆ alkyl;
R₂ₐ and R_{2b} are each independently H, D or C₁₋₆ alkyl;
R₄ and R₅ are each independently H, D or C₁₋₆ alkyl;
R₆ and R₇ are each independently H, D or C₁₋₆ alkyl;
R₈ and R₉ are each independently H, D or C₁₋₆ alkyl; or R₈ and R₉ together with the carbon atom connected thereto form C═O;
R₁₀ is C₁₋₆ alkyl, C₁₋₆ alkoxy, cyano or SR_{10d}, wherein the C₁₋₆ alkyl and the C₁₋₆ alkoxy are optionally substituted with 1 to 3 halogens;
R_{10d} is H, D or C₁₋₆ alkyl;
A is 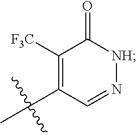 [SMILES: *c1cn[nH]c(=O)c1C(F)(F)F]
B is 5- to 6-membered carbocycle or heterocycle, wherein the heterocycle contains 1 to 3 heteroatoms selected from N, O and S;
m is 1, 2 or 3; and
n is 0, 1, 2 or 3.

9. The use-method according to claim 8, wherein the active ingredient is a compound of formula (I-3), or a pharmaceutically acceptable salt, a stereoisomer, or a deuterated compound thereof, wherein
X₁ is NH or 4- to 6-membered heterocycle containing 1 to 3 heteroatoms selected from N and O;
X₂ is O;
R₁ₐ and R_{1b} are each independently H, D or C₁₋₃ alkyl;
R₂ₐ and R_{2b} are each independently H, D or C₁₋₃ alkyl;
R₄ and R₅ are each independently H, D or C₁₋₃ alkyl;
R₆ and R₇ are each independently H, D or C₁₋₃ alkyl;
R₈ and R₉ are each independently H, D or C₁₋₃ alkyl; or R₈ and R₉ together with the carbon atom connected thereto form C═O;
R₁₀ is C₁₋₆ alkyl, cyano or SR_{10d}, wherein the C₁₋₆ alkyl is optionally substituted with 1 to 3 halogens;
R_{10d} is H, D or C₁₋₆ alkyl;
A is 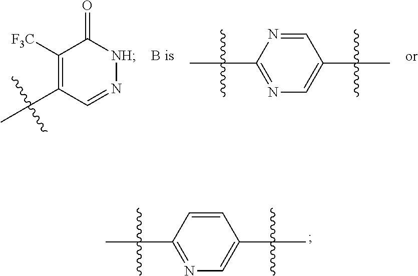 [SMILES: *c1cn[nH]c(=O)c1C(F)(F)F ; *c1cnc(*)nc1 ; *c1ccc(*)nc1]
m is 1, 2 or 3; and
n is 0, 1 or 2.

10. The use-method according to claim 9, wherein the active ingredient is a compound of formula (I-3), or a pharmaceutically acceptable salt, a stereoisomer, or a deuterated compound thereof, wherein
X₁ is NH;
X₂ is O;
R₁ₐ and R_{1b} are each independently H, D or C₁₋₃ alkyl;
R₂ₐ and R_{2b} are each independently H or D;
R₄ and R₅ are each independently H or D;
R₆ and R₇ are each independently H or D;
R₈ and R₉ are each independently H or D;
R₁₀ is CF₃;
A is 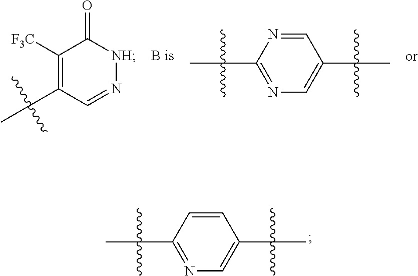 [SMILES: *c1cn[nH]c(=O)c1C(F)(F)F ; *c1cnc(*)nc1 ; *c1ccc(*)nc1]
m is 1, 2 or 3; and
n is 0, 1 or 2.

11. The method according to claim 1, wherein the active ingredient is selected from compounds as shown below, or a pharmaceutically acceptable salt, a stereoisomer, or a deuterated compound thereof:

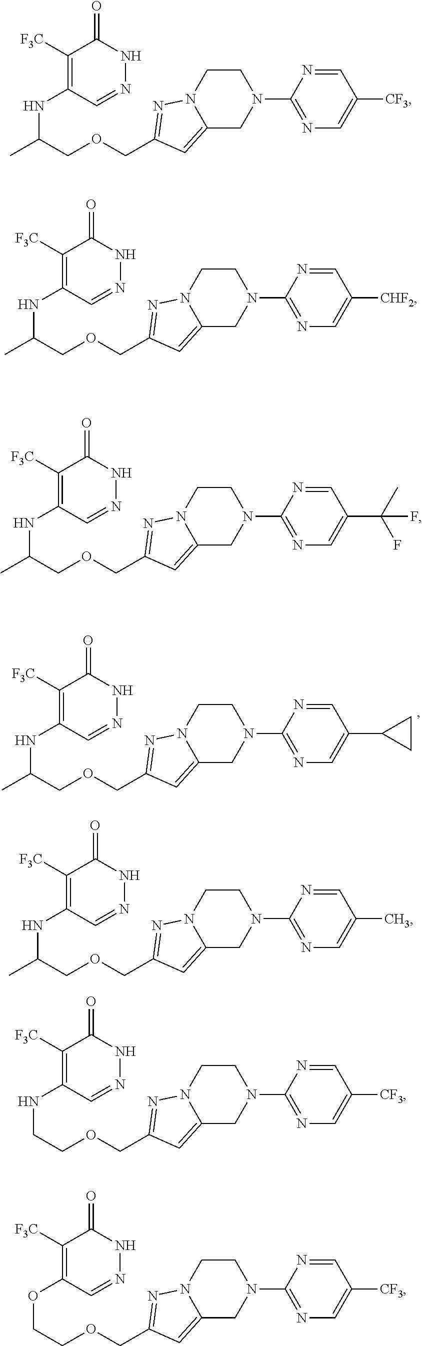

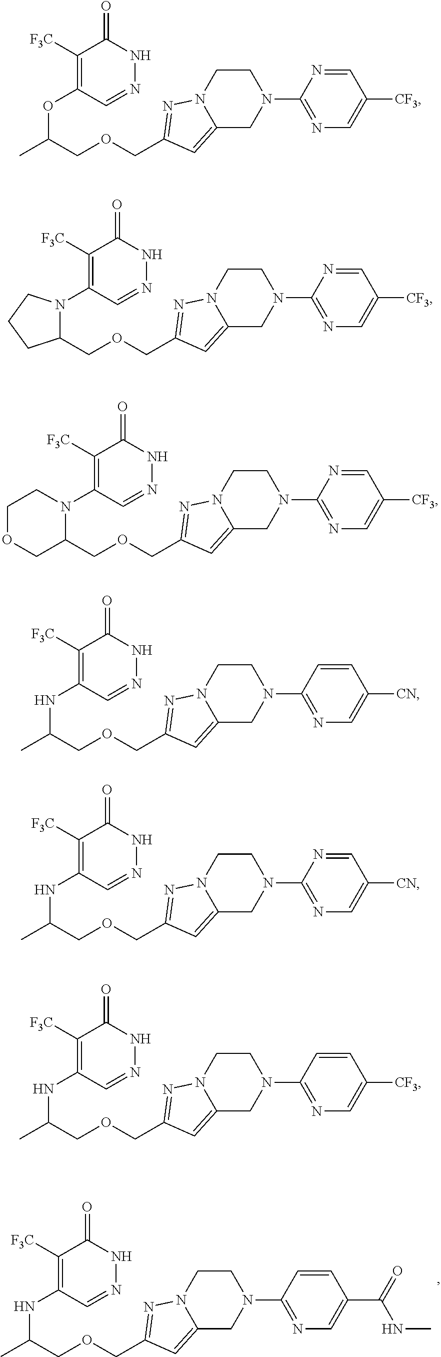

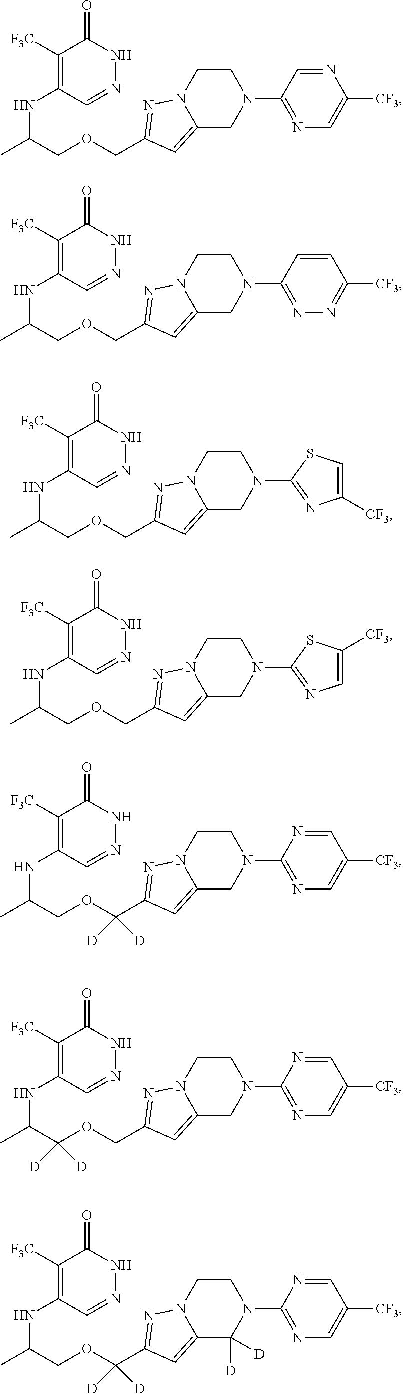

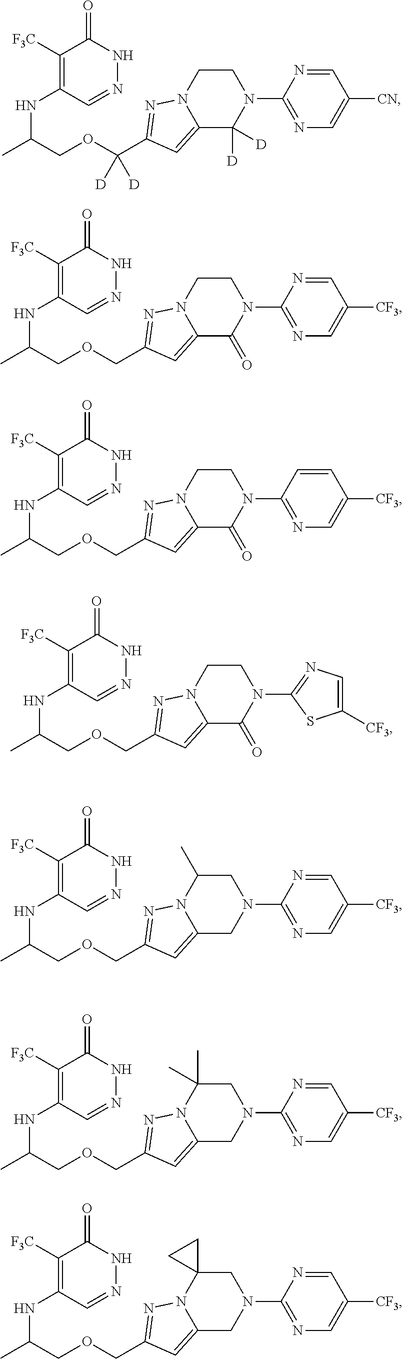

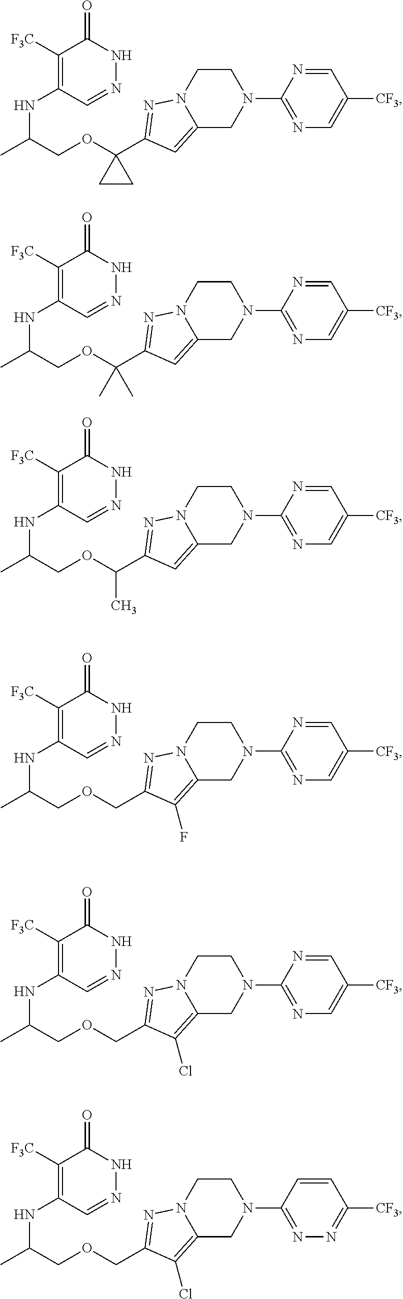

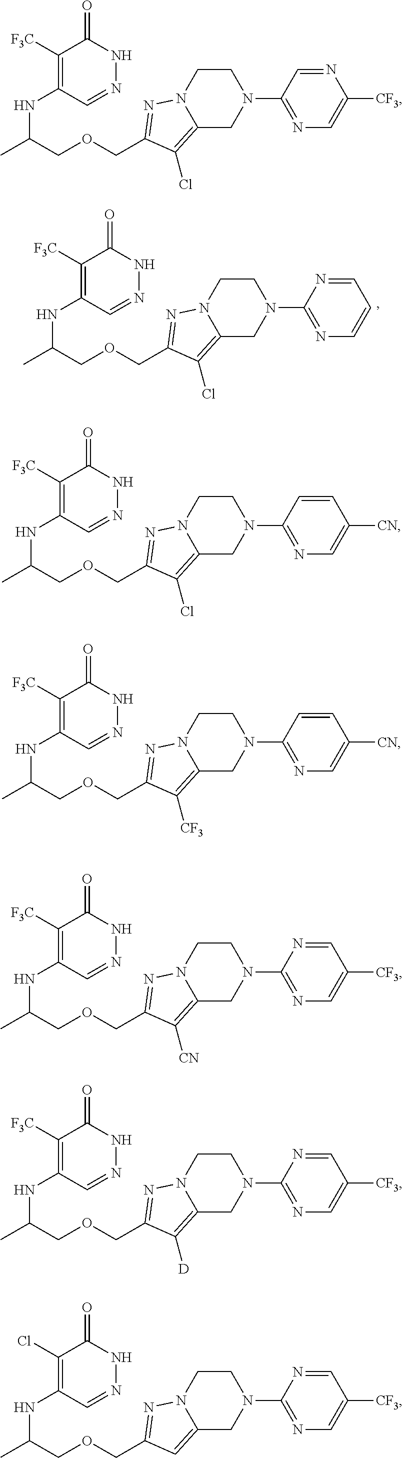

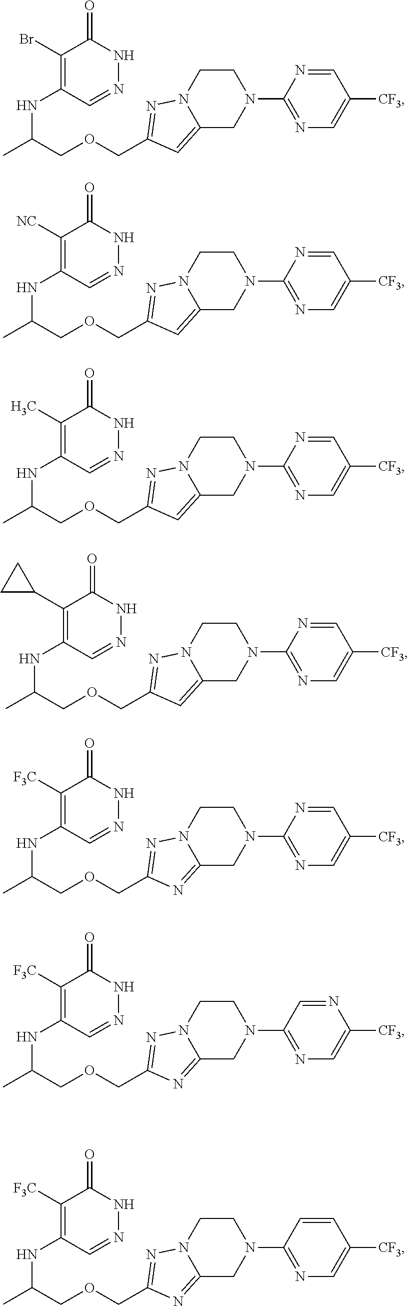

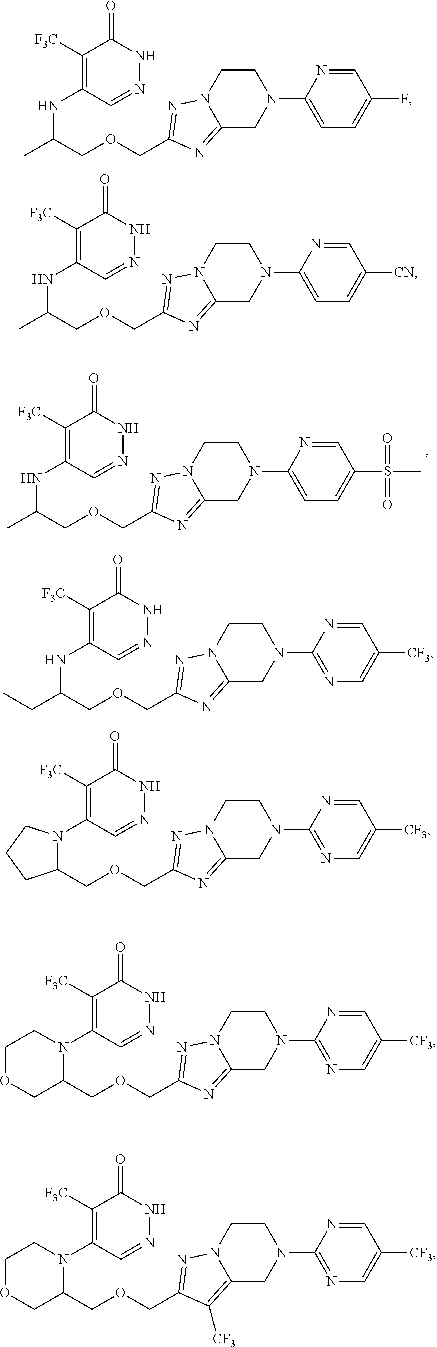

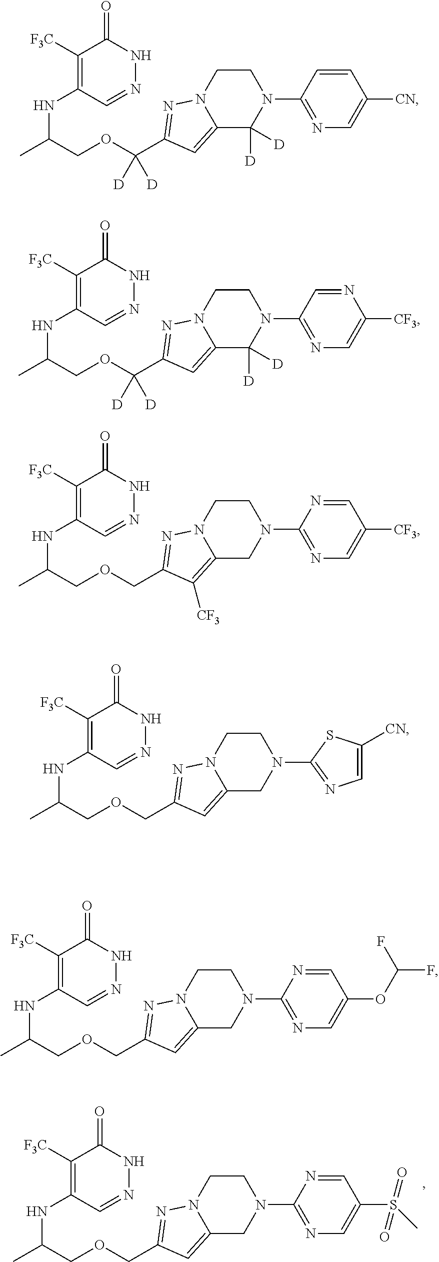

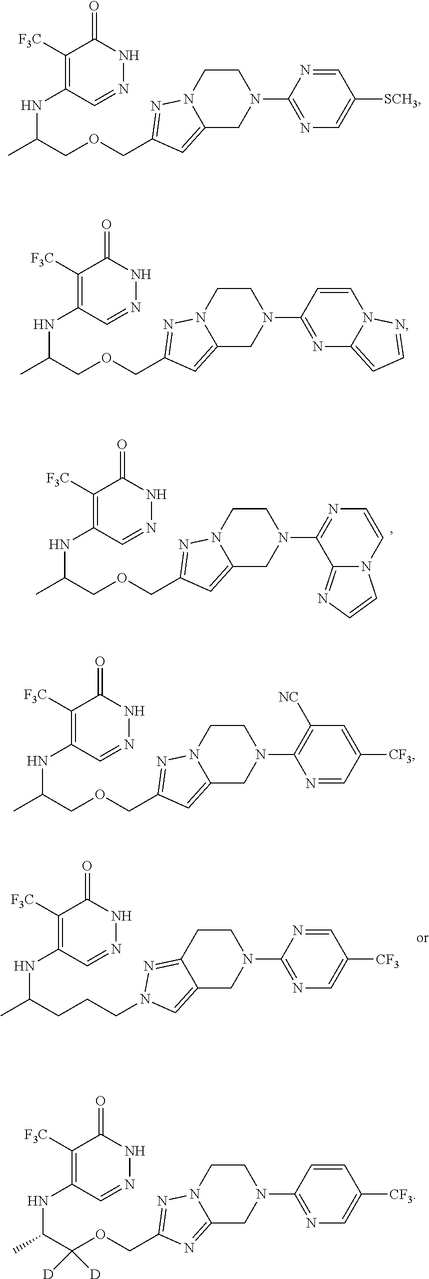 [SMILES: CSc1cnc(N2CCn3nc(COCC(C)Nc4cn[nH]c(=O)c4C(F)(F)F)cc3C2)nc1 ; CC(COCc1cc2n(n1)CCN(c1ccn3nccc3n1)C2)Nc1cn[nH]c(=O)c1C(F)(F)F ; CC(COCc1cc2n(n1)CCN(c1nccn3ccnc13)C2)Nc1cn[nH]c(=O)c1C(F)(F)F ; CC(COCc1cc2n(n1)CCN(c1ncc(C(F)(F)F)cc1C#N)C2)Nc1cn[nH]c(=O)c1C(F)(F)F ; CC(CCCn1cc2c(n1)CCN(c1ncc(C(F)(F)F)cn1)C2)Nc1cn[nH]c(=O)c1C(F)(F)F ; [2H]C([2H])(OCc1nc2n(n1)CCN(c1ccc(C(F)(F)F)cn1)C2)[C@H](C)Nc1cn[nH]c(=O)c1C(F)(F)F]

12. The method according to claim 1, wherein the active ingredient is contained in a pharmaceutical composition further comprising one or more pharmaceutically acceptable excipients, diluents or carriers.

13. The method according to claim 1, wherein the total daily dosage of the active ingredient is selected from 50-1500 mg, as measured by the amount of free base.

14. The method according to claim 1, wherein the active ingredient and the radiotherapy are administered simultaneously.

15. The method according to claim 1, wherein the active ingredient and the radiotherapy are administered sequentially.

16. The method according to claim 1, wherein the active ingredient is given orally at a frequency of twice daily or once daily.

17. The method according to claim 1, wherein the tumor is selected from solid tumor.

18. The method according to claim 13, wherein the total daily dosage of the active ingredient is selected from 100-1000 mg, as measured by the amount of free base.

19. The method according to claim 18, wherein the total daily dosage of the active ingredient is selected from 400-800 mg, as measured by the amount of free base.
